(19) United States
(12) Patent Application Publication
(10) Pub. No.: US 2011/0033560 A1
(43) Pub. Date: Feb. 10, 2011

(54) PREPARATION AND A COMPONENT INTENDED TO BE ADDED TO A TOBACCO PRODUCT

(75) Inventors: Osmo Suovaniemi, Helsinki (FI); Mikko Salaspuro, Helsinki (FI); Ville Salaspuro, Hus (FI); Martti Marvola, Helsinki (FI)

Correspondence Address: SUGHRUE MION, PLLC, 2100 PENNSYLVANIA AVENUE, N.W., SUITE 800, WASHINGTON, DC, 20037, US

(73) Assignee: BIOHIT OYJ, Helsinki (FI)

(21) Appl. No.: 12/678,037

(22) PCT Filed: Sep. 12, 2008
(86) PCT No.: PCT/FI2008/050507

§ 371 Date: May 18, 2010

(30) Foreign Application Priority Data
Sep. 14, 2007 (FI) 20070705

(51) Int. Cl. A61K 33/02; C07C 205/01; C07C 321/02; C07D 499/21; C07D 417/06; C07C 319/02; A24F 47/00; A61K 38/05; A61K 31/16; C07K 5/037; C07C 237/02; A61K 31/545; A61K 31/095; A61K 33/00; A61K 38/06; A61P 25/32; A61P 25/34; C01D 5/00; C01C 1/16

(52) U.S. Cl. 424/720; 562/553; 562/559; 540/311; 544/327; 568/62; 568/61; 131/270; 514/21.91; 514/626; 530/331; 564/197; 514/192; 514/706; 424/722; 514/21.9; 423/519; 423/470

(57) ABSTRACT

The present invention relates to the use of a preparation comprising a substance that is capable of binding acetaldehyde, and to the use of a filter that is attached to a tobacco product to reduce tobacco and/or alcohol dependence.

Smoking causes various effects that are detrimental to health. It increases, to an extremely significant extent, the risk of various cancers, such as cancer of the lungs, the mouth, the pharynx, the larynx, the oesophagus, and the stomach. Smoking causes coronary heart disease and chronic obstructive pulmonary disease. Smoking during pregnancy causes an increase in the rate of foetal and neonatal deaths, premature labour, and the low birth weights of new-horns. Children, whose parents smoke, suffer more often from bronchitis and pneumonia and have an increased risk of developing cancer later in their lives.

In spite of being well aware of the various severe health hazards, smokers find it extremely difficult to stop smoking. There are about 1.3 billion smokers in the world. Even if they tried to stop smoking by means of the nicotine replacement therapy, only about 20% would be successful in withdrawing from tobacco over a period of 12 months (Rose, 2006, Nicotine and nonnicotine factors in cigarette addiction. Nicotine Tob. Res. 3, 383-390 and Willemsen et al., 2003, Ned. Tijdschr. Geneeskd.) It has been estimated that smoking causes 5 million deaths a year in the world, mainly due to lung cancer, chronic obstructive pulmonary disease and cardiovascular diseases (Ezzati and Lopez, 2003, The Lancet 362: 847-852).

Alcohol is also known to cause cancer. According to Salaspuro (Salaspuro, M. Best Pract Res Clin. Gastroenterol (2003) 17:679-94) and Francheschi et al. (Cancer Res (1990) 50:6502-07), smoking and alcohol together, multiply the risks of developing cancers of the upper alimentary tract by 150-fold. It has been assessed that about 2 million people a year develop cancers of the upper alimentary tract, which are mainly caused by smoking and drinking alcohol.

Finding a method, composition or programme, which would enable the smokers to stop smoking and heavy users of alcohol to refrain from using alcohol, would be of an enormous consequence to the national health.

Acetaldehyde has been observed to cause cancer in animals and to also comprise a local carcinogen, when occurring in human saliva and the alimentary tract.

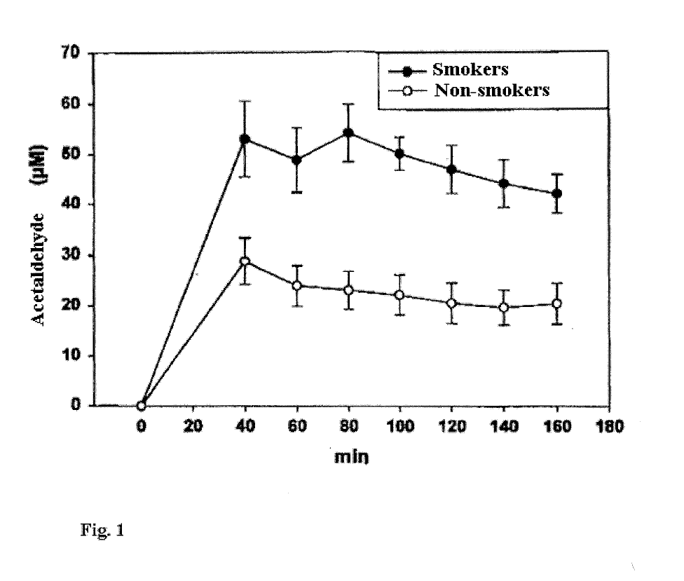

Salaspuro et al. 2004 (Salaspuro V, Salaspuro M. Synergistic effect of alcohol drinking and smoking on in vivo acetaldehyde concentration in saliva. Int J Cancer. 2004 Sep. 10; 111 (4): 480-3) have proven that the average content of acetaldehyde in saliva in vivo of smokers, even without smoking, after enjoying ethanol, is about two times higher than in non-smokers throughout an entire control period of 160 minutes (FIG. 1). The area under the graph that describes the acetaldehyde in the smokers' saliva was considerably larger than for the non-smokers, 114.8±11.5 μM×h compared with the value 54.2±8.7 μM×h, (p=0.002), respectively.

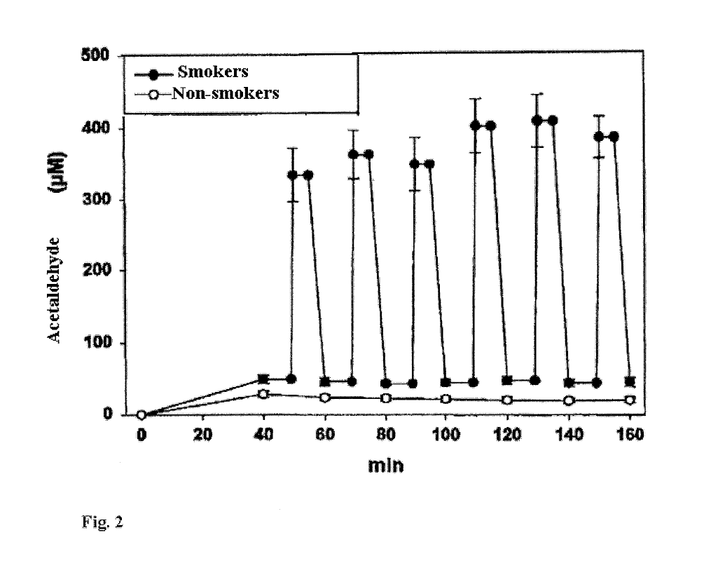

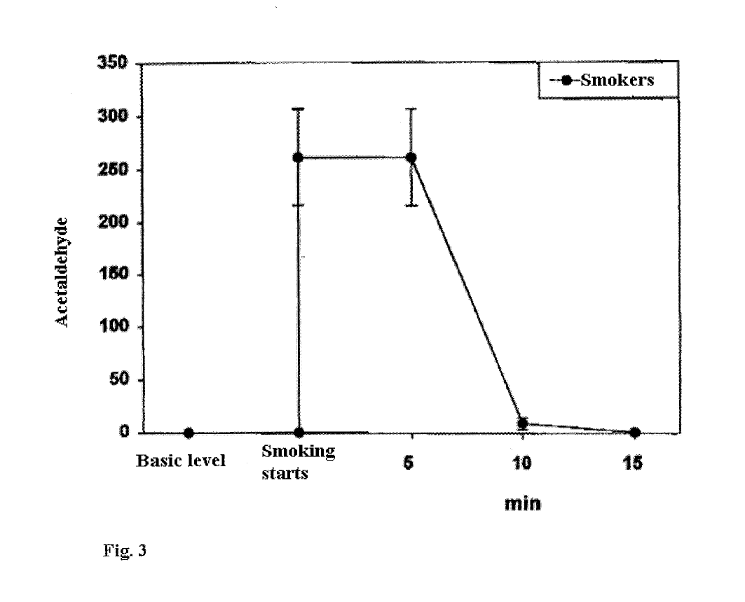

During a period of smoking a cigarette, the acetaldehyde in saliva in vivo increased by ten-fold from the levels that occurred when enjoying ethanol alone. The acetaldehyde in saliva immediately increased, when smoking began, but also quickly decreased after smoking stopped (FIG. 2). The area below the graph that describes the acetaldehyde in smokers was seven times larger when compared to the non-smokers, the difference being significant, 369.5±12.2 μM×h, respectively, compared with the value 54.2±8.7 μM×h, (p=0.001). During active smoking, the acetaldehyde in saliva increased from its basic level to a value of 261.4±45.5 μM. The acetaldehyde in saliva immediately increased, when smoking began, but also quickly decreased after smoking stopped (FIG. 3).

The first metabolism product of alcohol is acetaldehyde. Alcohol is evenly distributed in the liquid phase of the organism. Consequently, as long as an individual is enjoying alcohol and as long as there is alcohol in the organism, the amount of alcohol in the blood, saliva, gastric juice, and the contents of the bowel remain the same. The microbes of the alimentary tract are capable of oxidizing alcohol into acetaldehyde in the alimentary tract.

Acetaldehyde is formed in the organism from alcohol as a result of the hepatic metabolism and, locally, in the alimentary tract through microbes (Salaspuro et al, (1996) Ann Med 28:195-200). On the other hand, carcinogenic acetaldehyde can be formed endogenously by the microbes in the mouth from various foodstuffs, which have high sugar or hydrocarbon contents, particularly in an acid-free stomach. Atrophic gastritis and an acid-free stomach (achlorhydria) are well-known risk factors for the cancer of the stomach.

Due to the microbial metabolism, acetaldehyde is formed in the stomach in a case, where the stomach is acid-free or rendered acid-free by means of medicines (Väkeväinen et al., 2000, Alimentary Pharmacology Ther 14:1511-1518). In patients suffering from atrophic gastritis, microbes produce high acetaldehyde contents from ethanol and sugar in the stomach, resulting in an increased risk of developing cancers of the stomach (Väkeväinen et al, Scand J Gastroenterol 2002 (6):648-655).

No alcohol fermentation takes place in an acidic stomach. On the other hand, a Helicobacter pylori infection and certain medicines, such as the protein pump inhibitor (PPI) increase the pH of the stomach.

The patent application WO 02/36098 describes a preparation that slowly dissolves in the mouth, the stomach or the large intestine, releasing, over a long period of time, a substance that binds acetaldehyde and, in this way, reducing the risk of developing cancers of the mouth and the alimentary tract.

The patent application WO 2006/037848 describes preparations that are kept in the mouth or attached to a cigarette, or compositions, which are absorbed into or in some other way attached to the cigarette, the surface that is kept in the mouth, in particular, and which are used for the time it takes to consume the tobacco product. In that case, the acetaldehyde-binding substance is released into the saliva from the preparation that is kept in the mouth or from the tobacco product during the use of the tobacco product.

By means of the compositions and the preparations of the patent specifications mentioned above, it is possible to reduce the exposure of individuals to carcinogenic acetaldehyde.

Surprisingly, in connection with the present invention, it has been observed that the same or corresponding preparations, which release the acetaldehyde-binding compound into the mouth or the alimentary tract, can also be used for withdrawing from the use of tobacco and alcohol. In tests related to the present invention, it was observed that the testees no longer had a craving for tobacco, a pipe or cigars, when the acetaldehyde coming into the mouth during smoking and/or enjoying alcohol was bound. With the lack of the feeling of enjoyment, the testees gradually give up consuming cigarettes and alcohol.

The present invention provides a preparation and/or a component, such as a filter that is attached to the tobacco product, which can be used for withdrawing from tobacco and/or alcohol. The preparation or the component that is attached to the tobacco product removes an essential portion of the acetaldehyde that is present in the smoke of the tobacco product, or binds the acetaldehyde, which enters the saliva or the alimentary tract in connection with smoking or drinking alcohol.

The present invention also provides a method, by means of which a person can withdraw from tobacco and/or alcohol.

The method also preferably comprises a programme, which makes the withdrawal from tobacco and/or alcohol successful.

To be more precise, the use according to the invention is characterized by what is presented in the characterizing part of Claim 1, and the acetaldehyde-binding compound and the method are characterized by what is presented in the characterizing part of Claims 18 and 19.

FIG. 1 shows the salivary acetaldehyde in vivo after ethanol ingestion in smokers (without simultaneous smoking) and in non-smokers.

FIG. 2 shows the salivary acetaldehyde in vivo after ethanol ingestion in smokers (with simultaneous smoking) and in non-smokers. Differences between the acetaldehyde concentrations are significant at all time points from 40 to 160 min (p≦0.05).

FIG. 3 indicates the salivary acetaldehyde in smokers after smoking one cigarette (without simultaneous alcohol drinking).

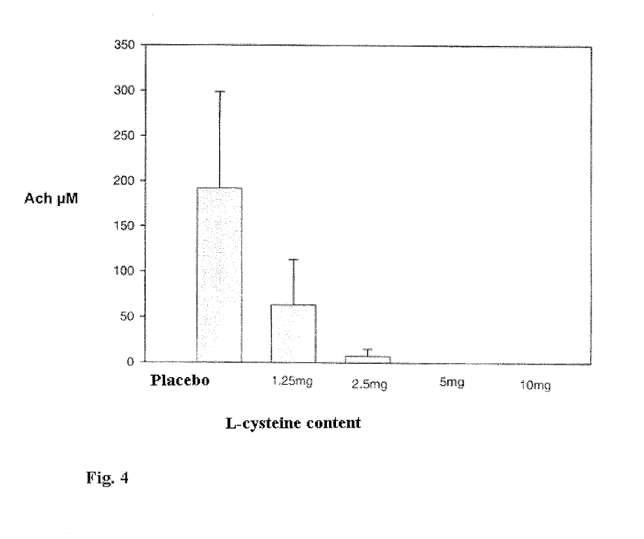

FIG. 4 shows the salivary acetaldehyde (SEM) after 5 min of smoking with a placebo, and with sucking tablets containing 1.25 mg, 2.5 mg, 5 mg or 10 mg of L-cysteine.

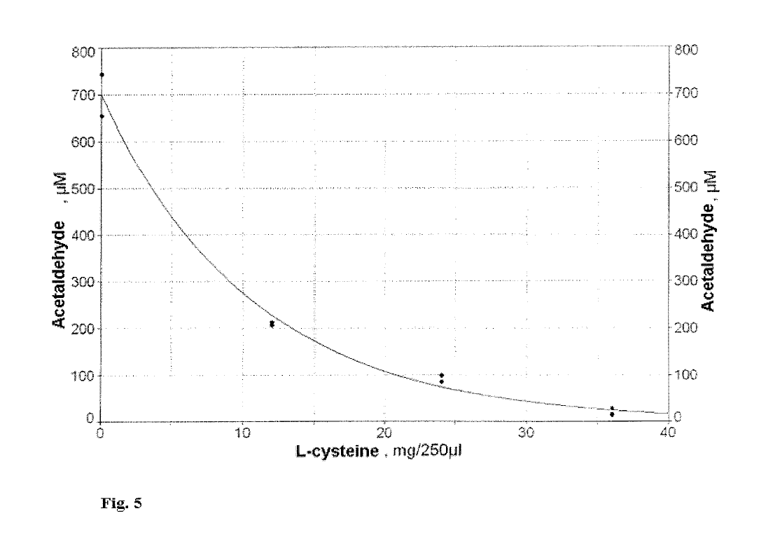

FIG. 5 shows the filtering effect of a filter, which has been moistened with a water-cysteine solution, on the concentration of carcinogenic aldehyde, i.e., acetaldehyde that occurs in cigarette smoke, in saliva during smoking. The use of the cysteine filter reduces the acetaldehyde concentration measured from the saliva during smoking to considerably below its limit value that is internationally determined for carcinogenity.

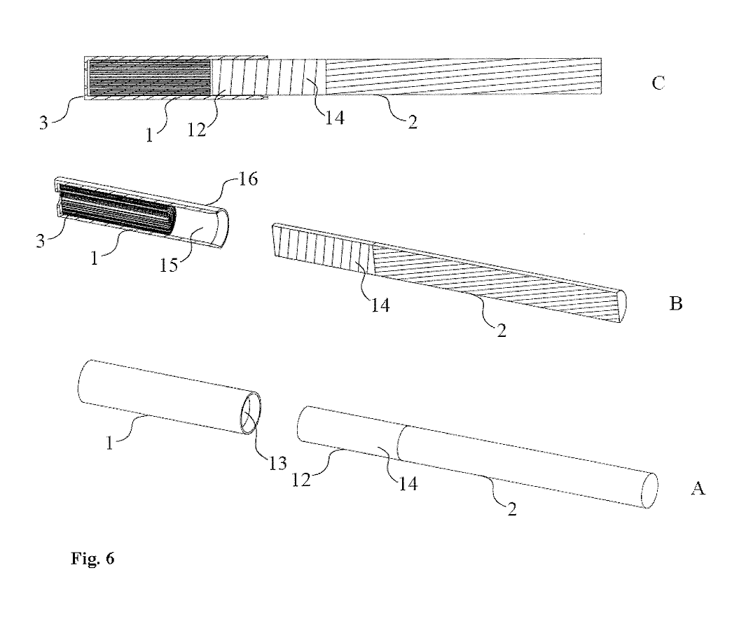

FIGS. 6A-C show a tobacco product and a component (a cigarette holder) that can be attached thereto (FIG. 6A) from above, and as a cross section from the side so that the component is detached from the tobacco product (FIG. 6B) or attached to the tobacco product (FIG. 6C).

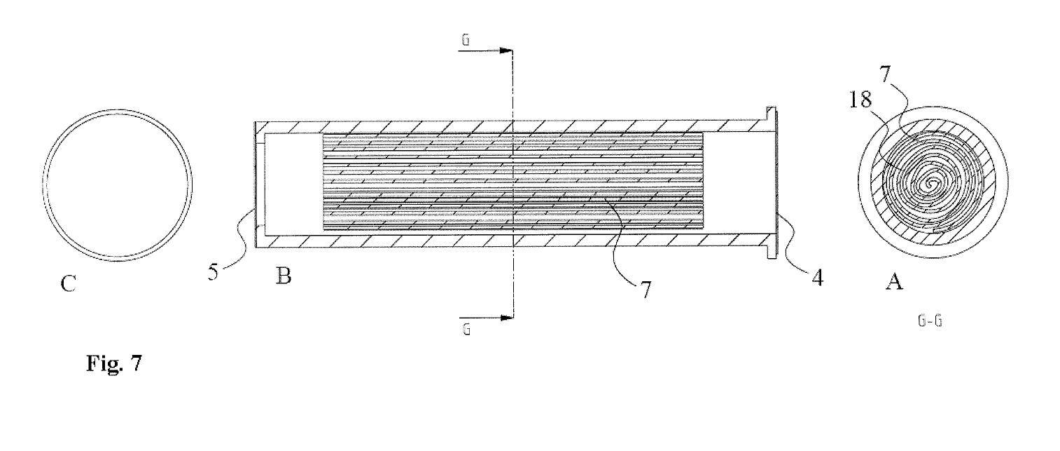

FIG. 7 shows a cross section of the component (the holder) as viewed from the end of the holder (FIG. 3A) and from the side (FIG. 3B). FIG. 3C shows the holder as viewed from the end of the holder.

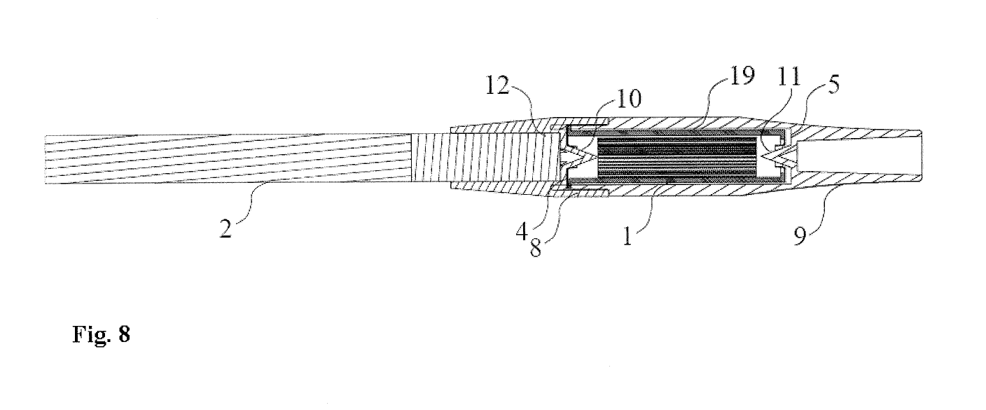

FIG. 8 shows a side view of a tobacco product, to which the component, such as a holder or filter that can be connected to the tobacco product, has been attached between attachments.

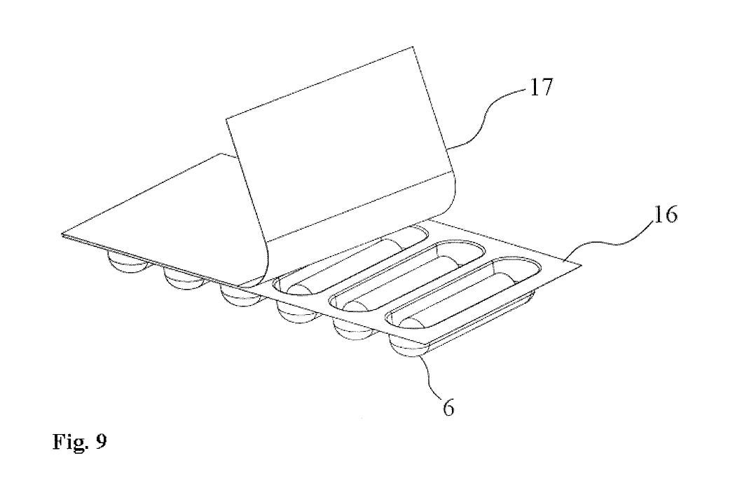

FIG. 9 shows a packaging plate, in which the components attachable to the tobacco product are packed.

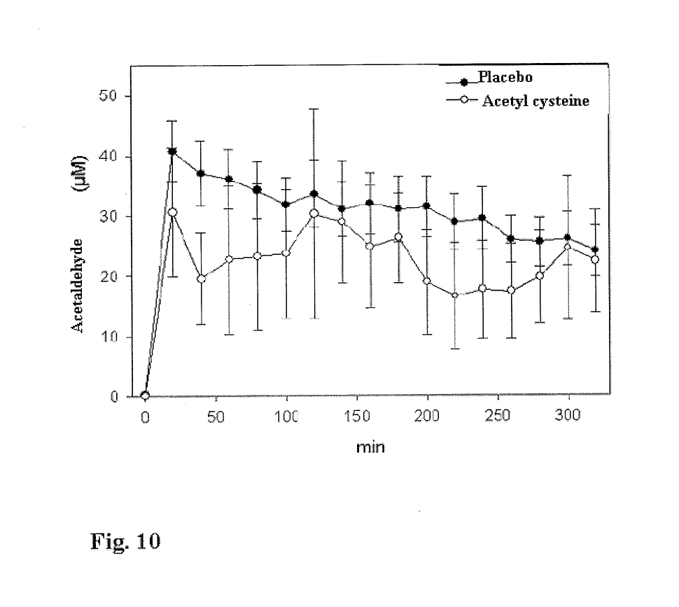

FIG. 10 shows the capability of acetyl cysteine to bind acetaldehyde compared with a placebo on testees, who had consumed alcohol.

### DETAILED DESCRIPTION OF THE INVENTION

The present invention provides preparations and tools, which can be used to reduce tobacco and/or alcohol dependence.

Tobacco or alcohol dependence or both can be reduced through the following stages:
a) An individual dependent on smoking and/or alcohol is given a composition containing a compound, which is capable of binding acetaldehyde, to be consumed simultaneously with consuming a tobacco product and/or alcohol or in connection with the same and/or,
b) an individual dependent on tobacco is given a tobacco product, to which a component, such as a filter is attached, to be attached to the tobacco product, the component containing the compound that is capable of binding acetaldehyde during smoking, and
c) an individual is allowed to smoke and/or drink alcohol, whereby the acetaldehyde-binding compound binds the acetaldehyde generated in the tobacco product or alcohol or both, thus preventing the formation of a substance called harmane, and
d) the stages a) and b), or both simultaneously, are repeated and the stage c) so many times that the reduction in smoking and/or alcohol dependence of the individual results in a cessation of smoking or consuming alcohol.

According to a preferred embodiment of the present invention, the stages a) and b), or both simultaneously, should be repeated in connection with smoking or consuming alcohol for at least 5, preferably at least 10, more preferably 15 to 50 times, or as many as 20 to 100 times, typically 10 to 20 times. It is preferable to repeat the stages sequentially without discontinuing the treatment or the programme by consuming tobacco products or alcohol as usual.

According to a preferred embodiment of the invention, one stage of the treatment or the programme comprises observing that smoking and/or alcohol dependence has reduced or ended. This can be carried out directly after undergoing the programme. If no withdrawal has taken place, the individual is advised to repeat the programme. In case withdrawal seems to have taken place, i.e., the individual no longer has a craving for tobacco and/or alcohol, checkups are carried out at regular intervals, first at intervals of 2 to 7 days, then, e.g., 2 weeks, and then one month. The withdrawal can be considered successful, if the individual has been able to refrain from regular smoking and/or regular and heavy alcohol consumption (exceeding moderate use) for the next half a year or preferably a year.

Furthermore, it is preferable to repeat the smoking and/or alcohol drinking activity by using the withdrawal preparation or the part or component, which is added to the tobacco product, in a way otherwise similar to the way the individual normally smokes or consumes alcohol. Extinguishing the craving for alcohol in normal drinking occasions by means of opiate antagonists is described in patent U.S. Pat. No. 4,882,335.

According to another preferred embodiment of the invention, the method also comprises a stage, where the individual is told about the health hazards and dependence caused by the acetaldehyde contained in tobacco and/or alcohol so that, when starting the method, they are aware of the damages caused by tobacco and/or alcohol.

The reduction in tobacco and alcohol dependence described in the present invention can be a consequence of the fact that, when binding the acetaldehyde into a form harmless to the organism, no products that cause dependence can be formed therefrom. According to Talhout et al., products, such as harmane and salsosinol, which are generated as condensation products from acetaldehyde and biogenous amines, may be substances that cause acetaldehyde dependence. Harmane is formed when smoking and the harmane levels in the blood of smokers are 2 to 10 times higher than those of non-smokers. Easily exceeding the blood-brain barrier and having enough potential to inhibit monoamine oxidase, harmane can affect the lower monoamine oxidase (MAO) activity observed in smokers' brains. Harmane and salsosinol inhibit monoamine oxidase (MAO). Talhout et al. have concluded that acetaldehyde may increase the potential of tobacco products to cause addiction, when acetaldehyde and biogenous amines are formed in cigarette smoke and/or in vivo. Talhout et al. 2007 have discovered that acetaldehyde causes dependence in rodents. This dependence cooperates with the dependence caused by nicotine.

McBride et al. (2002), in turn, have suggested that salsosinol enhance the craving for alcohol and, through that, the alcohol dependence. Naoi et al., NeuroToxicology 25 (2003) 193-204 and Haber et al. 1996, Alcohol Clin. Exp. Res. 20(1):87-92 have also discussed the effects of salsosinol.

Acetaldehyde may penetrate the blood-brain barrier and, on the other hand, it can also be formed in the brain. However, studies are controversial, as normal people do not have measurable amounts of acetaldehyde in their blood while burning alcohol. The brain studies, in turn, have been run on test animals and very high acetaldehyde concentrations were used.

The acetaldehyde, which is dissolved in saliva from tobacco or formed in saliva from alcohol as a combustion product of microbes, can condense with amino acid tryptamine and form harmane that functions as a neurotransmitter. Harmane, the condensation product of acetaldehyde and tryptamine, can also be formed in an acid-free stomach in connection with using drugs that inhibit the acid secretion and as a result of the action of the ADH enzyme contained in Helicobacter, whenever the individual either consumes alcohol or when alcohol drifts into the saliva or gastric juice through the blood circulation. Tryptamine occurs in milk products (e.g., fermented cheese) and soy products, for example. The harmane that is generated in the mouth or the stomach can absorb through the mucous membranes directly into the blood circulation and further drift to the brain without being subjected to the detoxication mechanisms of the liver in between. The harmane that enters the brain can cause dysphoric symptoms (resembling a hangover), or it may also enhance the alcohol dependence (Callaway et al. 1996, Life Sciences 58(21):1817-1821).

Binding acetaldehyde from the saliva or preventing it from entering the saliva considerably reduces the formation of harmane and any other wrong neurotransmitters and their entering the brain. The preparations and the tools according to the invention, and the use thereof, also contribute to reducing the symptoms of hangover.

The use of the products, tools, and methods according to the invention for withdrawing from tobacco and/or alcohol can be combined with the previously known methods that have been used for withdrawing from tobacco. Tobacco withdrawal products that typically contain nicotine, such as nicotine gum, can be used at the times, when the individual is able to refrain from smoking. Such products include, e.g., Nicorette ® products, such as chewing gums, tablets, plasters or products, which are described, for example, in the patent specifications U.S. Pat. No. 5,488,962 and U.S. Pat. No. 5,845,647. If the individual is unable to refrain from the use of tobacco products and restarts smoking, they can use preparations that contain the acetaldehyde-binding compounds according to the invention, or the parts or components, which are attached to the tobacco product and which contain the compounds capable of binding acetaldehyde.

The method according to the invention for withdrawing from tobacco and/or alcohol thus includes a stage(s), wherein the individual is withdrawn from the dependence caused by acetaldehyde by means of the method according to the invention; and a stage(s), wherein the individual is withdrawn from nicotine by means of tobacco withdrawal products that contain nicotine. The products and tools according to the invention are used during the time, when the individual is not able to refrain from smoking, and the nicotine-containing preparations during the time, when the individual is able to refrain from smoking.

The “tobacco product” refers to any tobacco product, such as a cigarette, cigar or pipe. The tobacco product can already include a filter that is normally used or the product can be without a filter. However, it is preferable that the filter does not prevent the smoke from drifting through the component (holder, acetaldehyde-binding filter) that contains the acetaldehyde-binding material.

“Smoking” refers to the use of the tobacco product, such as the cigarette, cigar or other tobacco product.

The “part or component that can be attached to the tobacco product” can also be called a cigarette holder or a filter. In that case, it refers to the acetaldehyde-binding filter as distinct from the conventional filters that are used in tobacco products.

“In connection with smoking” herein refers to the period of time that starts from starting to smoke and ends, when smoking stops. In particular, “in connection with smoking” herein refers to the period of time, when acetaldehyde enters the mouth of the smoker.

“In connection with consuming alcoholic drinks” herein refers to the period of time that starts from starting to consume alcohol and ends, when there is no more alcohol in the blood.

Binding of Acetaldehyde

“Binding of acetaldehyde” refers to a chemical reaction between the acetaldehyde and a compound that has a free amino group and/or sulphhydryl or sulphonic group, in which reaction the acetaldehyde together with the “acetaldehyde-binding substance” forms a larger molecule and in which reaction water can be formed. The “acetaldehyde-binding substance” preferably refers to a compound that comprises one or more free amino groups and sulphhydryl groups or sulphonic groups. The “compound” can refer to one or more compounds.

For example, when reacting with cysteine, acetaldehyde binds itself to both the sulphhydryl and the amino group, forming 2-methyl-L-thiazolidine-4-carboxylic acid and water. Acetaldehyde can bind itself to the amino group of almost any protein, whereby Schiff base or a 2-methyl-imidzole ring is formed.

Cysteine, its salts and derivatives are particularly suitable for the use according to the invention. The most suitable amino acids in the use according to the invention include L- and D-cysteine, acetyl cysteine, N-penicillamine or the derivatives of cysteine, which function in the same way as L- or D-cysteine and their salts. The compound is most suitably L-cysteine and its salts.

Suitable compounds for binding acetaldehyde in the organism also include the compounds according to the formula (I):

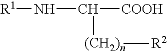

wherein

R¹ is hydrogen or an acyl group that has 1 to 4 carbon atoms;

R² is a sulphhydryl or sulphonic group;

n is 1, 2, 3 or 4.

The amino acids or other compounds that suitably bind acetaldehyde and contain a free sulphhydryl (SH) and/or amino (NH₂) group include:

L-cysteine

D-cysteine

cysteinic acid,

cystine,

cysteine-glycine,

threo- or erythro-β-phenyl-DL-cysteine,

β-tetramethylene-DL-cysteine,

methionine,

serine,

D-penicillamine and its N-terminal dipeptides,

semicarbazide,

glutathione,

reduced glutathione,

β-mercaptoethyl amine

D,L-homocysteine,

DL-homocysteinic acid

N-acetylcysteine,

L-cysteinyl-L-valine,

β-β-tetramethylene-DL-cysteine,

cysteinylglycine,

mercaptoethyl glycine,

cysteine hydrochloride,

thiamine hydrochloride,

sodium metabisulphite,

arginine,

glycine,

lycine,

ammonium chloride,

1,4 ditiothreitol,

mercaptane,

or a salt of any of these compounds.

The effects of some acetaldehyde-binding or other aldehyde-binding substances can be improved by vitamins, such as L-ascorbic acid.

Suitable compounds to be used in the invention also include the salts of the acetaldehyde-binding compounds (pharmaceutically acceptable salts, in particular).

Any acetaldehyde-binding compounds that present no health hazards in the dosages used are suitable for the preparations according to the invention.

The acetaldehyde-binding compounds disclosed by the present invention can also be used to bind aldehydes other than acetaldehyde.

It is also advantageous, if the taste or the smell of the compounds is not unpleasant or too strong. It is possible to disguise the unpleasant taste of an effective compound by means of suitable sweetening agents or flavourings; however, by using compounds that have a mild and/or pleasant taste, the compound can be kept simple and it is easier to produce. Another way of reducing the significance of the product's taste is to use as small amounts as possible.

Tobacco can be used by smoking, chewing, or wetting or snuffing. According to our research, smoking, in particular, seems to cause the formation of acetaldehyde in the mouth. Smoking in connection with the present invention typically refers to smoking cigarettes or cigars or, alternatively, a pipe.

A “harmful/carcinogenic concentration of acetaldehyde” in the human mouth, oesophagus, stomach and large intestine is 50 to 100 μMol/l of saliva. Even lower acetaldehyde concentrations together with tryptamine cause the formation of harmane. A detrimental, carcinogenic or harmane-forming acetaldehyde concentration in the human mouth can be obtained, for example, in connection with smoking and/or drinking alcohol, and even after drinking alcohol as long as there is alcohol in the blood, saliva or gastric juice.

An object of the present invention is to keep the acetaldehyde in the mouth on a level essentially lower than without using the preparations according to the present invention. The acetaldehyde concentration of saliva is preferably kept on a level that is at least 60%, and more preferably at least 80% lower than without using the composition. It is most preferable to remove essentially all of the acetaldehyde so that there is no time to form the harmane that causes addiction. In connection with smoking, all of the acetaldehyde in saliva can be removed by the preparations mentioned above. In connection with using alcohol, the acetaldehyde that is formed in the saliva can preferably be reduced by at least about 70%. Preparations that slowly release cysteine into the stomach can be used to preferably reduce the acetaldehyde by at least about 70%, preferably by at least about 80%, and more preferably by at least about 90%.

It should be noted that the products described in the present invention can either be used alone or together; e.g., the holder capable of binding acetaldehyde and the preparation that is kept in the mouth can thus be used simultaneously.

The “local preparation that is placed in the mouth” refers to all preparations that are sucked or chewed in the mouth or that can be placed between the cheek, the lip or the tongue and the gum (gingiva), and wherein the release of the substance is intended to have a local effect in the mouth. The preparation preferably also has an effect in the pharynx, oesophagus, or stomach. The preparation can release the acetaldehyde-binding substance in a short time, e.g., during 5 to 15 minutes, or for a long time, such as more than a half an hour.

Furthermore, the local preparation refers to capsules, tablets or other preparations that contain the acetaldehyde-binding compound that is released into the stomach (or later on into the intestines, such as the large intestine). The compound can be inside the capsules in the form of granules, for example. These preparations are intended for removing the acetaldehyde that drifts into the stomach along with the saliva or that is generated in an acid-free stomach or a stomach infected by the Helicobacter by microbes from alcohol. The same is also true for the use of medicines that prevent the secretion of acid, together with alcohol. Suitable preparations are described, for example, in the published patent applications WO 02/36098 and PCT/FI2007/050287 and PCT/FI2007/050288.

The term “composition” herein refers to a composition that comprises the effective substance(s), possibly mixed together with a suitable carrier. The composition can be in the form of a local preparation, which is suitable to be used in the mouth, the stomach (or later on in the alimentary tract, such as the large intestine).

The “composition” refers to a non-toxic composition suitable for human consumption, which can be used as an additive of foodstuffs, for example. The composition also refers to a pharmaceutically acceptable composition that contains pharmaceutically acceptable carriers. The effective compounds also refer to the salts of these compounds; particularly salts that are suitable for human consumption or pharmaceutically acceptable. The compositions according to the invention are particularly suitable for oral use.

The local preparation according to the invention can be selected from among chewable or sucking tablets, buccal tablets, sublingual tablets, candies, pastilles, chewing gums, bubble gums, gels, medicine tablets or capsules and medicine granules.

In addition to what is called an effective substance(s) that binds acetaldehyde, the preparation preferably comprises at least one carrier, which does not prevent but facilitates the release of the effective substance. In the case of a preparation that slowly releases the effective substance, it is preferable that the carrier adjusts the release of the effective substance. Furthermore, it is preferable that the preparation has a shape that makes it easier to keep in the mouth either during smoking or when drinking alcohol. The preparation can otherwise be of any shape, such as round or elliptical, longitudinal, capsule-shaped, convex or annular. It is also preferable that the preparation is relatively small so that its use does not complicate or change the smoking activity or the use of alcohol.

The preparation can be placed in the mouth during smoking or consuming alcohol or it can be attached to the tobacco product in a suitable way. The preparation can be kept attached to the tobacco product during smoking or it can be detached from the tobacco product and placed in the mouth, when smoking begins.

The ideal operating time of the preparation that is used in connection with smoking is the same as that consumed by smoking (about 5min). The operating time of the preparation, which is used in connection with consuming alcohol and which slowly releases the acetaldehyde-binding compound either into the mouth or the stomach, is preferably 2 to 4 hours.

It is advantageous, if the amount of effective substance can be kept as small as possible, as the taste of the compound does not then need to be disguised at all or needs to be disguised to a minor degree only, if the taste of the substance is unpleasant. The individual using the composition does not need to consume exceedingly high concentrations of the compound. The preparation is also less expensive.

The preparation according to the present invention preferably comprises 1 to 300 mg, more preferably 1 to 250 mg, more preferably 1 to 200 mg, even more preferably 1 to 150 mg, most preferably 1 to 100 mg of the acetaldehyde-binding substance. Larger amounts are particularly advantageous, when the purpose is to bind acetaldehyde in the mouth or the stomach over a long period of time, or if the purpose is to bind the acetaldehyde that is formed from both tobacco and alcohol. Larger amounts of effective substance can be used in the stomach, in particular, where the taste of the acetaldehyde-binding compounds causes no problems. In that case, the dosage unit of the composition preferably contains 50 to 500 mg of the acetaldehyde-binding substance; the amount of acetaldehyde-binding substance is more preferably 50 to 300 mg, and most preferably 100 to 200 mg. The preparations that release cysteine into the stomach can typically contain 200 to 500 mg of the acetaldehyde-binding substance per capsule.

According to a preferred embodiment of the invention, the preparation according to the invention, the one used in the mouth, in particular, comprises 1 to 50 mg, more preferably 5 to 30 mg, even more preferably 5 to 10 mg, or as much as 1 to 5 mg, typically 10 to 20 mg or 1 to 20 mg, and in some embodiments 15 to 20 mg of the acetaldehyde-binding substance(s). The amount of substances can preferably be larger, if the preparation is kept attached to the tobacco product during smoking compared to placing the preparation in the mouth, when smoking begins.

In addition to the preparations described above, the scope of the present invention also includes other preparations and compositions, which are used with tobacco products and which are capable of releasing the acetaldehyde-binding substances into the saliva during smoking. For example, the patent specification WO 2006/037848 describes compositions, which are kept in the mouth and which release the acetaldehyde-binding compound during the use of the tobacco product. The composition that comprises effective substance(s), can also be concentrated, for example, and/or dried, and/or impregnated into the tobacco product, the filter or the holder, as described in the published patent application mentioned above.

An impregnated filter can also be separately from the tobacco product and it can, for example, be attached to the tobacco product or placed in the holder of the tobacco product, as described in the patent application WO 2006/037848.

The amount of acetaldehyde-binding substances in these applications can preferably be larger than in the preparation that is kept in the mouth. The amount of acetaldehyde-binding substances can be larger than 5 mg, preferably larger than 10 mg, more preferably larger than 20 mg, most preferably larger than 30 mg, even more preferably larger than 50 mg per one tobacco product or filter or holder. Smaller amounts are preferable, if the composition has been concentrated and/or dried and/or impregnated on the surface of the filter, the tobacco product or the holder only.

In addition to the effective substance(s), the composition can comprise:

1. Pharmaceutically acceptable diluents (fillers, extenders),

2. Sweeteners, such as sugars and sugar alcohols,

3. Flavourings, and

4. Slip additives/lubricants.

The sugars can comprise, for example, saccharose, fructose or glucose or mixtures thereof. The sugar alcohols can comprise mannitol, sorbitol, maltitol, lactitol, isomalt, or xylitol or mixtures thereof. No additive preferably reacts with the other ingredients in the preparation. Not being too sweet, a preferable sweetener comprises mannitol, and its amount in the preparation can be quite large; accordingly, it functions as a diluent at the same time.

The flavourings can comprise, for example, spearmint, peppermint, menthol, citrus fruit, eucalyptus or aniseed or a mixture thereof.

The preparation can also comprise other ingredients, such as substances that prevent bad oral smell, substances that function as breath fresheners and/or prevent dental caries, or the preparation can comprise vitamins. The preparation can also comprise substances that increase salivation. However, these additives should not prevent the quick release of the acetaldehyde-binding substance into the saliva. As previously described herein, the preparation should release the acetaldehyde-biding substance so effectively that an essential amount of acetaldehyde is bound to the saliva before the acetaldehyde influences the cells of the mucous membrane in the mouth.

According to a preferred embodiment of the present invention, the preparation (such as one tablet) can essentially comprise or consist of the following:

| | |
|---|---|
| Acetaldehyde-binding substance(s) | 1 to 50 mg |
| Diluting agent(s)/sweetener(s) | 50 to 750 mg |
| Flavouring(s) | q.s. |
| Lubricant(s) (0.5 to 3% by weight) | 5 to 25 mg |

The preparation can be a sucking tablet comprising:

| | |
|---|---|
| Acetaldehyde-binding substances | 1 to 50 mg |
| Sugar or sugar alcohol, such as mannitol | 50 to 750 mg |
| Flavouring | q.s. |
| Magnesium stearate | 5 to 25 mg |

The composition is prepared by mixing a powdery mass and compressing it into sucking tablets by any well-known methods.

If the amount of acetaldehyde-binding compounds is increased, the amount of diluent(s)/sweetener(s) and flavourings can also be increased, as the taste of the acetaldehyde-binding substances must be disguised.

According to another preferred method of the present invention, the preparation can essentially comprise or consist of the following:

| Acetaldehyde-binding substances | 1 to 50 | mg |
|---|---|---|
| Gum base comprising sweeteners or other substances | 500 to 1500 | mg |
| Flavouring | q.s. | |
| Lubricant (0.5 to 3% by weight) | 5 to 30 | mg |

The gum base, which can comprise medicated chewing gum (Morj aria, Y. et al., Drug Delivery Systems & Sciences, vol. 4, No. 1, 2004) that comprises natural or synthetic elastomers, softeners, waxes and lipids. Natural gum bases, including crude rubber and smoked natural rubber, are permitted by the FDA. However, modern gum bases are mostly synthetic and include styrene-butadiene rubber, polyethylene and polyvinyl acetate. The gum base constitutes 15 to 40% of the chewing gum. The remaining portion consists of medicine, sugar, sweeteners, softeners, flavourings and colouring agents. The majority of the chewing gum-based drug delivery systems are prepared using conventional methods. However, directly compressible powder gums are modern alternatives to the medicated chewing gums. Pharmagum is a compressible new gum system. It is a mixture of polyol(s) and/or sugars with a gum base. A formulation that contains Pharmagum gums can be compressed into a gum tablet by using conventional tablet presses. The manufacturing method is quick and inexpensive. The amount of gum base in the preparation, comprising sweeteners can be 50 to 500 mg, preferably 500 to 1500 mg.

Pharmagum S contains rubber base and sorbitol, Pharmagum M contains rubber base, mannitol, and isomalt.

The preparation can be a chewing gum comprising:

| Acetaldehyde-binding substances | 1 to 50 | mg |
|---|---|---|
| Pharmagum S | 500 to 1500 | mg |
| Flavouring | q.s. | |
| Magnesium stearate (0.5 to 3% by weight) | 5 to 30 | mg |

The composition is prepared by mixing a powdered mass and compressing it into chewable tablets.

The preparation can be a buccal tablet comprising:

| | |
|---|---|
| Acetaldehyde-binding substances | 1 to 50 mg |
| Non-ionized macro molecules | 5 to 25 mg |
| Ionizing macro molecules | 2 to 10 mg |
| Flavouring(s) | q.s. |
| Lubricants | 0.5 to 3% by weight |

The non-ionized macro molecules include, e.g., methylcellulose (MC), hydroxypropyl cellulose (HPC) and hydroxypropyl methylcellulose (HPMC), and polyethylene glycol (PEG). The ionizing polymers include, e.g., sodium carboxymethyl cellulose (NaCMC), alginic acid, sodium alginate, chitosan, polycarbofile (Noveon™) and carbomer (Capropol™).

The preparation can also be a sublingual tablet essentially comprising or consisting of the following:

| | |
|---|---|
| Acetaldehyde-binding substances | 1 to 50 mg |
| Diluent(s)/sweetener(s) q.s. | 50 to 500 mg |
| Flavouring(s) | q.s. |
| Lubricants | 0.5 to 3% by weight |

The diluents include, e.g., lactose, calcium phosphates, starch, carboxymethyl cellulose, hydroxymethyl cellulose. The sweetener can be, for example, mannitol or xylitol.

According to a preferred embodiment of the invention, the preparations according to the invention are provided in a tool kit comprising:
Numerous cigars or cigarettes, and
numerous preparations that comprise the acetaldehyde-binding substance(s) in amounts that are capable of binding acetaldehyde from the saliva during smoking essentially to a level, where the acetaldehyde was before smoking.

The preparation is preferably capable of binding acetaldehyde from the saliva during the smoking of at least one, possibly 1, 2 or 3 cigarettes or cigars.

The tool kit can comprise a tobacco package or box intended for the cigars or cigarettes, which is connected to another box or package intended for the preparations. The cigars or cigarettes and the preparation can be in the same or a separate package or box. Two packages or boxes can be separate or combined. The tool kit preferably comprises essentially the same or a larger number of preparations than cigars and cigarettes. The number of cigars or cigarettes in the package is preferably at least 10, typically at least 20, in some cases at least 30, typically 20 to 40 or 20 to 50 cigars or cigarettes.

The number of cigars or cigarettes in the package is preferably at least 10, typically at least 20, in some cases at least 30, typically 20 to 40 or 20 to 50 cigars or cigarettes.

According to another preferred embodiment of the invention, the preparation can be attached to a tobacco product, such as a cigar, cigarette, holder or pipe. The preparation can be in any suitable form, such as a chewable or sucking tablet, buccal tablet, sublingual tablet, candy, pastille, medicine tablet, chewing gum, capsule, granules or gel. The preparation can also be of any suitable shape, such as round, elliptical, convex, nail-shaped, cylindrical, capsule-shaped, annular or rectangular.

According to a further preferred embodiment of the invention, the preparation can be attached to the cigar, cigarette, holder or pipe detachably. The individual starting to smoke can detach the preparation from the tobacco product by hand, teeth or in some other way, and chew, suck or hold the preparation in the mouth, e.g., under the tongue or between the cheek and the gum (gingiva), so that the preparation stays in contact with the saliva.

Administering the Acetaldehyde-Binding Substance

The content of acetaldehyde formed in the saliva as a consequence of smoking can be reduced so that, in connection with smoking, the preparation, preferably one or two preparations at the same time, are placed in the mouth, under the tongue or in the cheek, or between the cheek and the gum, for example, releasing, at a suitable and preferably constant velocity, cysteine or another acetaldehyde-binding substance that essentially has the same effect as cysteine, continuously and advantageously, until one tobacco product has been consumed. When starting the next tobacco product, a new acetaldehyde-binding preparation is placed in the mouth. According to a preferred embodiment of the invention, the preparation is also capable of reducing the salivary acetaldehyde content during smoking one cigar, cigarette or pipe, to the level where the acetaldehyde was before smoking.

The use of the acetaldehyde-binding substance is repeated as many times as a new tobacco product is started. It is preferable to place the preparation in the mouth already before starting to smoke a new cigar, cigarette or pipe.

The preparation according to a preferred embodiment of the present invention is capable of releasing the acetaldehyde-binding substance into the saliva under the conditions prevailing in the mouth within less than 30 minutes, and preferably within less than 15 minutes from the point of time, when the preparation is brought into contact with the saliva. Accordingly, the acetaldehyde-binding substances are released within 0 to 5 minutes, more preferably within 0 to 10 minutes, most preferably within 0 to 15 minutes from the point of time, when the preparation is brought into contact with the saliva. The release of the acetaldehyde-binding substances preferably takes essentially the time of smoking one cigar or cigarette, i.e., the time of actual smoking and a few minutes longer.

Preparation that Acts in the Mouth for a Long Time

During smoking or drinking alcohol, compositions can be used, which slowly release the acetaldehyde-releasing substance into the mouth and which are described in the patent application WO02/36098. “Prolonged release of the effective substance” means that the substance is released over at least 30 minutes, preferably at least 120 minutes, most preferably more than four hours. Using the compositions according to the invention, release times of the effective substance of as long as 4 to 8 hours can be achieved. The compound is preferably released under the conditions of the mouth in amounts of 15 to 25 mg an hour. 1 or 2 preparations according to the invention can be placed in the mouth at a time and they can be replaced with new ones at 4 to 10-hour intervals, most preferably at 6 to 8-hour intervals.

Preparation that Acts in the Stomach

“A long-acting preparation that has a local effect on the stomach” refers to all monolithic or multi-particular tablets or capsules or granules as such, which, when wetted by the gastric juice, adhere to the mucous membrane of the stomach or form a gel that floats in the contents of the stomach, or which have a shape or size that prolongs their residence time in the stomach, thus enabling a prolonged release into and a local effect of the drug on the stomach. For example, the long-acting preparation that locally acts on the stomach can also be a liquid preparation (mixture), which is taken orally and the physical structure of which is a gel. Preparations that have a local and long-term effect on the stomach are described in the patent specification WO 02/36098, for example.

The preparation that acts in the stomach preferably comprises a non-toxic carrier that contributes to the prolonged release of the effective substance into the stomach. The prolonged release refers to the release of the effective substances under the conditions of the stomach for at least 30 minutes. The effective substances are preferably released during 0.5 to 8 hours, typically 2 to 6 hours, normally 2 to 4 hours.

According to some preferred embodiments of the invention, the dosage of the preparation can be renewed at 4 to10-hour intervals, preferably at 6 to 8-hour intervals.

The composition that acts in the stomach can be in the form of a preparation, which is a tablet, capsule, granule, powder, or a tablet or capsule that contains granules or powder. The composition can be in the form of a monolithic or multiple-unit preparation, such as a tablet or capsule or granule.

An individual dosage of the preparation can comprise a tablet or capsule or a suitable amount of granules, or a tablet or capsule containing granules or powder.

It is preferable that the preparation be in a form that has a diameter of at least 7 mm, preferably 8 to 15 mm, and more preferably 11 to 15 mm. This helps the preparation to stay in the stomach for a sufficient time to ensure the slow release of the effective substances.

The amount of substances released from the preparation into the stomach is preferably 40 to 80 g in an hour.

The purpose of the carrier in the preparation is to provide a prolonged release of the effective substance under the conditions of the stomach.

According to a preferred embodiment of the invention, the composition comprises a carrier that does not dissolve or dissolves poorly under the conditions of the stomach. Alternatively, the composition can be coated with a film insoluble in water.

According to another embodiment of the invention, the carrier can form, in the stomach, a gel that floats in the contents of the stomach.

According to a third embodiment of the invention, the composition can be in the form of a liquid preparation, which is taken orally (an oral mixture) and the physical form of which is a gel.

According to a fourth embodiment of the invention, the preparation can stick to the mucous membrane of the stomach.

According to a preferred embodiment of the invention, the composition comprises a carrier that does not dissolve under the conditions of the stomach. The carrier can be, for example, a polymer, such as methacrylate polymer, e.g., Eudragit RS or S, or ethylcellulose.

The composition can comprises substances, which have been selected from a group comprising one or more acetaldehyde-binding compounds, a polymer that does not dissolve in the stomach, and filler.

The composition preferably comprises 1 to 40% by weight, preferably 5 to 40% by weight, more preferably 10 to 30% by weight of the acetaldehyde-binding compound(s). Typically, the amount is 20 to 25% by weight.

The composition preferably comprises 10 to 50% by weight, preferably 20 to 40% by weight, and more preferably 20 to 30% by weight of polymers.

The composition preferably comprises 20 to 70% by weight, more preferably 40 to 60% by weight, even more preferably about 50% by weight of fillers.

According to a preferred embodiment of the invention, the composition comprises:

Matrix granules that do not dissolve in the stomach. The composition may comprise, for example:

| | |
|---|---|
| Acetaldehyde-binding substance(s) | 5 to 40% by weight |
| | (preferably 25 w-%) |
| Polymer insoluble in the stomach | 10 to 50% by weight |
| | (preferably 20 to 30% by weight) |
| Inert filler | 20 to 70% by weight |
| | (preferably 40 to 60% by weight) |
| Ethanol | q.s. |

The polymer that does not dissolve in the stomach can be any additive generally used in the pharmaceutical industry, such as methacrylate polymer, e.g., Eudragit RS or S, or ethylcellulose (EC). The inert filler can be, for example, dicalcium hydrogen phosphate, microcrystalline cellulose (MCC), or another corresponding non-swelling substance. The solid substances are mixed and moistened with ethanol. The moist mixture is granulated using the methods and equipment that are generally used in the pharmaceutical industry. The dried granules can be used as such or they can be portioned out into doses, such as capsules.

According to a preferred embodiment of the invention, the composition comprises matrix tablets that do not dissolve in the stomach. The composition can comprise, for example:

| | |
|---|---|
| Acetaldehyde-binding substance(s) | 5 to 40% by weight, |
| | (preferably 25 w-%) |
| Polymer insoluble in the stomach | 10 to 50% by weight |
| | (preferably 20 to 30% by weight) |
| Inert filler | 20 to 70% by weight |
| | (preferably 20 to 50% by weight) |

The polymer that does not dissolve in the stomach can be any additive generally used in the pharmaceutical industry, such as methacrylate polymer, e.g., Eudragit RS or S, or ethylcellulose (EC). The inert filler can be, for example, dicalcium hydrogen phosphate, microcrystalline cellulose (MCC), or another corresponding non-swelling substance. The solid substances can be mixed and the mixture granulated using, for example, ethanol or a hydrophilic polymer solution. The granules are compressed into tablets by the methods and equipment that are well known in the pharmaceutical industry. The release of the effective substances is now based on the diffusion of the water-soluble effective substances from the pores formed in the tablet matrix.

According to a preferred embodiment of the invention, the composition can be protected so that the effective substances are not released into the mouth. The granules, tablets, and capsules can be coated with a water-soluble film, which also effectively covers or disguises the taste of the acetaldehyde-binding compounds.

According to another preferred embodiment of the invention, the composition comprises substances that are selected from a group comprising: one or more acetaldehyde-binding compounds, water-soluble filler(s), and a substance(s) that form a porous film for coating the preparation.

The composition preferably contains 1 to 50% by weight, preferably 5 to 40% by weight, more preferably 20 to 50% by weight, and more preferably 20 to 30% by weight of the acetaldehyde-binding substance(s). Typically, the amount is about 20 to 25% by weight.

The composition preferably comprises 10 to 80% by weight, preferably 40 to 80% by weight, and more preferably 50 to 60% by weight of filler(s).

The composition preferably comprises substances, such as ethylcellulose and hydroxypropyl methylcellulose, which form the porous film. The ratio of EC to HPMC can be 3/2-7/3.

The preparation, preferably a tablet, is coated with a film that does not dissolve in the stomach. The composition can comprise, for example:

| | |
|---|---|
| Acetaldehyde-binding substance(s) | 1 to 50% by weight, |
| | (preferably 20 to 50% by weight) |
| Water-soluble filler(s) | 50 to 80% by weight |
| | (preferably 30 to 60% by weight) |
| Substance(s) that form the | q.s. |
| porous film | |

The water-soluble filler can be, for example, lactose or some other water-soluble filler commonly used in the pharmaceutical industry. The solid substances are mixed and the mixture is compressed into tablets by means of the methods and equipment commonly used in the pharmaceutical industry. The porous film can be manufactured from a water-soluble polymer, such as hydroxypropyl methylcellulose (HPMC) and from a polymer insoluble in water, such as ethylcellulose (EC). The relative portions of the film-forming substances, such as EC and HPMC, can comprise 2 to 5 portions of EC and 1 to 2 portions of HPMC. Under the conditions of the stomach, the water-soluble polymer dissolves and pores are formed in the polymer that is insoluble in water. The release of the effective substances is now based on the diffusion of the water-soluble effective substances through the pores formed in the film. The film-forming substances also effectively disguise or conceal the taste of the acetaldehyde-binding compounds.

According to a preferred embodiment of the invention, the preparation can be coated with the HPMC film or it can be inside a hard gelatine or HMPC capsule or some other preparation.

The preparation may comprise the substances intended for a preparation for binding acetaldehyde in the stomach. Optionally, the preparation may be in the form of a capsule, such as HPMC capsule or gelatine, particularly hard gelatine.

As acetaldehyde is also formed in the large intestine, for example, in connection with drinking alcohol, in particular, it is preferable to protect the composition so that the effective substances or some of them are not released until into the large intestine. Suitable preparations are described in the patent specification WO 02/36098, for example.

Useful enteric polymers in these preparations include, e.g., the hydroxypropyl methylcellulose phthalate grades, the hydroxypropyl methylcellulose succinate grades or the hydroxypropyl methylcellulose acetate succinate (HPMC-AS) grades or the like, which are sold by the trade name Agoat™, particularly Aqoat AS-HF™, the cellulose acetate phthalate (CAP) grade that is sold by the trade name Aquateric™, and a methyl acid derivative, methacrylic acid methyl methacrylate copolymers, the grades that are advantageously sold by the trade name Eudragit-S™, in particular.

Suitable preparations are described, for example, in the patent applications PCT/F12007/050287 and PCT/FI2007/050288.

Component Attached to the Tobacco Product

According to the invention, the compounds that are capable of binding acetaldehyde are absorbed as an aqueous solution into or, in some other way, attached to the material, which is packed into the component that is intended to be attached to the tobacco product. When the component is tightly attached to the suction head of the tobacco product, cigarette smoke travels through the component, and the material inside the component, containing the compounds capable of binding acetaldehyde, binds the acetaldehyde from the cigarette smoke so that the content of acetaldehyde cannot increase to a harmful level in the mouth and the saliva. As the compounds capable of binding acetaldehyde bind the acetaldehyde in an aqueous phase, the material containing the acetaldehyde-binding compounds should be moist enough when smoking begins in order for the binding reaction to take place.

The purpose of a preferred embodiment of the present invention is to provide a solution, in particular, due to which the capability of the component attached to the tobacco product to bind harmful acetaldehyde remains during the storage of the product.

According to a preferred embodiment of the invention, the compounds capable of binding acetaldehyde are absorbed as an aqueous solution into a porous material, the purpose being to keep the water content of the material high enough until the filter with its material is used. The high enough water content herein means that the acetaldehyde-binding compounds in the material remain active, i.e., are capable of binding the acetaldehyde into a form harmless to health.

According to a preferred embodiment of the invention, the moisture in the material contained in the component that is attached to the tobacco product is preserved by means of moisture barriers. The component that is attached to the tobacco product is partly or fully protected by means of the moisture barriers.

According to another preferred embodiment of the invention, the filter material containing the acetaldehyde-binding compounds is packed into a container that can be added inside the component that is attached to the tobacco product. The container can be partly or fully protected by means of the moisture barriers.

By means of the filter capable of binding aldehydes according to the invention, at least 60%, typically at least 80%, preferably at least 85%, more preferably at least 90%, and most preferably at least 95% of the aldehydes contained in the smoke of a cigarette, cigar, pipe or other tobacco product can be bound.

The use of the acetaldehyde-binding filter is simple. The component containing the filter material is attached to the tobacco product in the same way as the mouthpieces or cigarette holders on the market. According to a preferred embodiment of the invention, the component is fully or partly protected by means of moisture barriers. The moisture barriers are removed from the ends of the component or the component is removed from its package that works as a moisture barrier. The filter is immediately ready to be used. In the embodiments, where the filter material contained in the component contains a sufficient amount of acetaldehyde-binding compounds, the component can be used during more than one use (time of smoking) of the tobacco product.

According to another preferred embodiment of the invention, a container containing the acetaldehyde-binding filter material is added to the component, such as a mouthpiece or holder, before use. The container is preferably partly or fully protected by means of moisture barriers. Before use, the moisture barriers are removed and the container is inserted into the component that is attached to the tobacco product.

The solutions according to the invention are now considered by means of FIGS. 6 to 9.

FIG. 6 shows a component 1 according to the invention that is attached to a tobacco product, and a tobacco product 2. In FIGS. 6A and 6B, the component 1 and the tobacco product 2 are apart, in FIG. 6C they are attached to each other. In FIG. 6A, the component 1 and the tobacco product 2 are shown from above; in FIGS. 6B and 6C as cross sections viewed from the side. The component 1 that is intended to be attached to the tobacco product comprises a cylindrical space 13, which is open at both ends and essentially hollow on the inside, the component being tightly adaptable to the suction head 12 of the tobacco product 2. When fitted into the tobacco product, both ends of the component 1 should be open so that the smoke drifts through the component during smoking. The component 1 can also be called a cigarette holder or a filter. The tobacco product shown in FIG. 2 also contains a conventional filter 14.

The space 13 inside the component 1 that is intended to be attached to the tobacco product contains porous material 3, into which an aqueous solution containing at least one acetaldehyde-binding compound has been absorbed, or to which at least one aldehyde-binding compound has been attached in some other way. The material containing the acetaldehyde-binding compounds can herein also be called filter material. At the end of the component 1, which is adaptable to the suction head 12 of the tobacco product, there is an empty space 15 that is free from material 3. When fitting the component 1 to the tobacco product 2, the suction head 12 of the tobacco product fits inside the empty space 15 at the end of the component 1 so that the edge 16 of the fitting area covers the end 12 of the tobacco product.

After manufacturing the filtering part of the component, the material 3 should be moist. The material is preferably moistened with 20 to 500 μl, more preferably 50 to 250 μl of water, or if the acetaldehyde-binding compounds are contained in an aqueous solution, with the aqueous solution containing the acetaldehyde-binding substance. The amount of aqueous solution, which is needed to moisten the material, naturally, depends on the length of the component (the holder) and the amount of material. The water content of the material 3 in the component 1 can be kept essentially unchanged by means of a moisture barrier(s) 4, 5, which can be opened or removed before smoking begins.

According to another preferred embodiment of the invention, the filter material is moistened before use. In that case, however, it should be ensured that the acetaldehyde-binding compounds in the filter material are able to preserve their capability to react with the acetaldehyde even when drying. According to a preferred embodiment of the invention, the acetaldehyde-binding compounds are attached to the filter material in an aqueous solution and the moisture of the filter material is kept essentially unchanged until the tobacco product and the component containing the filter material that is attached thereto are used.

FIG. 7 shows a moisture barrier 4, 5 according to a solution according to the invention. It is manufactured from an essentially airtight material, such as aluminium foil. The moisture barrier 4, 5 covers both ends of the component 1.

According to a preferred embodiment of the invention shown in FIG. 8, the component 1 can be provided with attachments 8, 9, which contain a projection 10, 11 that pierces the moisture barrier 4, 5, when the attachments are in place. The attachments can be placed in the suction head 12 of the tobacco product 2 so that the component 1 remains between the attachments 8, 9, which can be tightly joined to each other. After the moisture barriers 4, 5 have been pierced open by means of the projections 10, 11; smoke is able to drift through the tobacco product 2 and the component 1.

FIG. 9 shows a packaging sheet 16 according to a preferred embodiment of the invention for packaging the components 1. The component 1 is packed into a recess 6 of the packaging sheet 16 that is made of a formable material, such as plastic, the recess preferably having a shape that conforms to the shape of the component 1. The recess 6 can be covered with a moisture barrier 17, whereby the component 1 is left in the airtight space of the recess 6. The component 1 can be pushed out of the recess 6 so that the moisture barrier 17 is torn or the moisture barrier 17 can be pulled away from the component. The same packaging sheet 16 can have several components 1 packed in respective recesses 6.

The porous material 3, which the acetaldehyde-binding compound is absorbed into or attached to in some other way, is packed inside the component 1 so that as large as possible a surface becomes in contact with the smoke. FIG. 7 shows a way of packaging according to a preferred embodiment of the invention. According to the embodiment, the material 7 is wound into the form of a roll and packed inside the component 1. In that case, the smoke is able to drift between the slightly spaced apart layers 18 of the material 7, and the acetaldehyde-binding compound in the material gets in contact with the acetaldehyde in the smoke. According to another preferred embodiment of the invention, cellulose can be packed as a wadding-type mass inside the component 1.

Typically, the component that can be attached to the tobacco product has an elongated shape of a cigarette holder/mouthpiece.

Inside the component 1, there is preferably a container 19, inside of which the porous material 3 and the acetaldehyde-binding compounds are packed. The container is preferably comprised of a material similar to plastic. The container preferably has such a shape and size that it fits inside the component 1. The outer dimensions of the container can be, for example, 6-10 mm×30-40 mm, typically 8 mm×35 mm.

According to a preferred embodiment of the invention, the container 19 is packed, and separately partly or fully protected by means of the moisture barriers. The container can be inserted into the component 1, such as a cigarette holder or mouthpiece, which is to be attached to the tobacco product, before smoking starts.

The porous material can comprise cellulose, for example. The cellulose can be, for example, cellulose wadding or filter paper or another material that behaves in a corresponding manner. The amount of material per one cigarette holder or container inside the same can be 50-200 mg, preferably 70-150 mg, typically 90-120 mg. The amount of porous material depends on the size of the mouthpiece or the cigarette holder or the container inside the same, and also on how large an amount of acetaldehyde-binding compounds are to be attached to the material.

The amount of aqueous solution used for one cigarette holder or the container inside the same is preferably 20-400 μl, typically 50-250 μl. The amount of water should be sufficient to keep the acetaldehyde-binding compounds active.

The component, such as the cigarette holder or the container inside the same, which is intended to be attached to the tobacco product, preferably contains the acetaldehyde-binding compound in an amount, which is sufficient to bind the acetaldehyde contained in the smoke of at least one tobacco product. This amount is preferably 0.5 mg-100 mg, typically 10-50 mg. A larger amount is preferable, particularly, when the component, such as the cigarette holder or container, is used more than once.

It should be noted that the acetaldehyde-binding compounds can also bind other aldehydes occurring in cigarette smoke, and the preparations, filters, and holders according to the invention are also suitable for binding other aldehydes than acetaldehyde.

“Aldehydes” refer to C₁-C₇ aldehydes, which can be hydrocarbon chains with a linear, branched or cyclic structure. In addition to the aldehyde group, they can also contain other reactive groups as well as double or triple bonds between the atoms. Low-molecular aldehydes include formaldehyde (C₁), acetaldehyde (C₂), and acrolein (C₃) and crotonaldehyde (C₄), the latter two containing a double bond. Of these, acetaldehyde is important, in particular. Aldehydes herein refer to the aldehydes that occur in the smoke of the tobacco products.

The aqueous solution that contains the acetaldehyde-binding compound also refers to aqueous solutions, which have been buffered, their acid content has been adjusted and/or to which salts have been added. The binding of acetaldehyde and their filtering from the cigarette smoke can be improved, for example, by buffering or adjusting the acid content in a slightly alkaline or acidic direction by adding small amounts of salts to the aqueous solutions.

Although in the following, a reference is made to acetaldehyde, in particular, the reference also stands for other aldehydes that occur in cigarette smoke.

According to the invention, the compounds that are obtained from aldehydes; particularly acetaldehyde, by chemically binding are harmless to the organs.

### Examples

### Example 1

A sucking tablet was prepared, comprising:

| | |
|---|---|
| Cysteine | 20 mg |
| Mannitol (or an equivalent sugar or sugar alcohol) | 750 mg |
| Flavouring | q.s. |
| Magnesium stearate | 10 mg |

The composition was prepared by mixing a powdery mass and compressing it into sucking tablets.

### Example 2

Sucking tablets were prepared as in Example 1, but they comprised 1.25 mg, 2.5 mg, 5 mg, and 10 mg of cysteine.

### Example 3

A chewing gum was prepared, comprising:

| | |
|---|---|
| Cysteine | 20 mg |
| Pharmagum S, M or C | 1000 mg |
| Flavouring | q.s. |
| Magnesium stearate | 20 mg |

The composition was prepared by mixing a powdery mass and compressing it into chewing gums. Another composition was prepared, comprising 500 mg of Pharmagum S or M, and 20 mg of magnesium stearate.

### Example 4

A buccal tablet was prepared, comprising:

| | |
|---|---|
| Cysteine | 20 mg |
| Methocel | 25 mg |
| Carbopol | 7 mg |
| Flavouring | q.s. |
| Magnesium stearate | 2 mg |

The composition was prepared by mixing a powdery mass and compressing it into buccal tablets.

### Example 5

A sublingual tablet was prepared, comprising:

| | |
|---|---|
| Cysteine | 10 mg |
| Mannitol | 250 mg |
| Flavouring | q.s. |
| Magnesium stearate | 5 mg |

The composition was prepared by mixing a powdery mass and compressing it into sublingual tablets.

### Example 6

Two individuals tested the preparation prepared according to Example 1. The salivary acetaldehyde contents of the testees were measured before smoking and then after 5 minutes during smoking, i.e., 0 min, 5 min, 10 min, and 15 min after the testees started smoking. Each testee smoked one cigarette and, at the same time, saliva was collected from their mouths, as they sucked placebo tablets. Smoking lasted for 5 min. In another test, the testees repeated the study by sucking tablets containing 20 mg of cysteine.

Before smoking, the salivary acetaldehyde content of each testee was very low. In the second test, the acetaldehyde content had reduced to a non-measurable level already after the first 5 minutes.

### Example 7

Five smokers (of the age of 29±2.8) participated in the study, in which three cigarettes were smoked (with cleaning periods in between). While smoking each cigarette (in 5 minutes time), the voluntaries sucked tablets blindfold, containing a placebo, 1.25 mg, 2.5 mg, 5 mg, 10 mg or 20 mg of L-cysteine. The acetaldehyde in the saliva samples was analysed by gas chromatography after 0, 5, 10, 20 minutes from starting to smoke.

The L-cysteine tablets (5 mg, 10 and 20 mg) removed from the saliva all of the acetaldehyde originating from tobacco (see FIG. 4). The average salivary acetaldehyde contents immediately after smoking were 191.2±48.5 μM, 0 μM, 0 μM, 0 μM with the placebo and the 5 mg, 10 mg, and 20 mg L-cysteine tablets, respectively.

The study showed that even 5 mg of L-cysteine, when delivered with a melting tablet, completely inactivated the carcinogenic acetaldehyde in the saliva during smoking. The L-cysteine tablet of 1.25 mg reduced the amount of acetaldehyde by about two thirds compared with the placebo.

### Example 8

Sucking tablets, chewing gum, buccal tablets, and sublingual tablets were prepared, comprising 5 mg of L-cysteine.

### Example 9

Moistened cysteine filter

The materials used in the tests

Tobacco:

Marlboro Red, Amer Tupakka

North State Plain, BAT

The entire filter of Marlboro Red was removed or, in some tests, a 2-3 mm piece of the original filter was left in the cigarette.

The Cigarette Holder

In the tests, the ready commercial cigarette holders/mouthpieces of Denicotea, www.denicotea.de, were used. In the cigarette holders/mouthpieces, plastic cylinder-shaped “containers” were used, in which the filter material was packed. The original content (intended for the removal of nicotine) of the containers was removed and replaced with the material that was used in the study. The outer dimensions of the container were: diameter 8 mm and length 35 mm.

The Filter Material

Cellulose wadding (c. 100 mg), filter paper (Whattman),

Commercial filters: Smart (the importer: Altadia Finland Oy) and Ventti (Philip Morris Finland Oy),

Cysteine

Cysteine was dissolved in distilled water, 30-180 mg of L-Cysteine/1 ml. The manufacturer: Fluka, 30089.

Test Arrangements:

The filter material that had been packed in plastic cylinders was moistened with the water-cysteine solution (50-250 μl), and the filter container thus made was placed in a Denicotea cigarette holder. After this, the testees smoked the cigarettes that had been placed in the mouthpieces. During smoking, saliva samples were collected from those participating in the tests and the acetaldehyde in the saliva was analyzed by gas chromatography, Perkin-Elmer, HS40, headspace Gas Chromatograph.

Results:

Similar results were obtained with both tobacco brands.
The filter material that had been moistened with the water-cysteine solution removed almost all acetaldehyde from the saliva (95%), FIG. 5.
The acetaldehyde contents measured from the saliva in connection with using the cysteine filter decreased to considerably below the international limit value 100 μM set for the carcinogenicity of acetaldehyde.
The filterability was dependent on the amount of cysteine; FIG. 5 shows the effect of the moistened cysteine filter on the salivary acetaldehyde content during smoking.

The results show that:
The filter material alone does not filter the acetaldehyde.
The filter moistened with water does not filter the acetaldehyde.
The filter and cysteine in solid form do not filter the acetaldehyde.
The results can be generalized to various tobacco brands, with or without a filter.
Commercial filters do not bind water; therefore, they are not suitable for the filter material.
It is not preferable to use the cysteine filter together with the original filter of the cigarette. In that case, the smoke does not flow normally and easily through the two filters. A small part of the original filter can be preserved, if the material of the cysteine filter is porous enough.

### Example 10

The testees enjoyed alcohol in amounts of 0.8 g per kilo of body weight. Thereafter, the testees attached, under their upper lip, a buccal tablet that contained 100 mg of N-acetyl cysteine and that slowly released the acetyl cysteine. The salivary acetaldehyde levels were measured at intervals of 20 minutes up to 320 minutes. The results are shown in FIG. 10. Throughout, the acetaldehyde contents of those using the acetyl cysteine tablets were lower than of those using the placebo.

### Example 11

The testees are allowed to smoke a cigarette or a cigar and simultaneously keep in their mouth a chewing gum that contains at least 0 and 5 and 10 mg of cysteine. Smoking is repeated with the cysteine chewing gum in the mouth 5, 10, 15, 20, and 30 times. The testees report on their observations concerning the craving for tobacco every time they start a new cigarette. During the test, the testees do not smoke cigarettes or cigars without the acetaldehyde-binding preparation. They will report on their smoking habits at 2-week intervals for a period of one year. If they have started smoking again, they are advised to start using cysteine chewing gum every time they smoke.

### Example 12

The test arrangement according to claim 11 is repeated so that the acetaldehyde-binding holder and the cysteine preparation are used simultaneously in the mouth. The testees will report on their smoking habits as in the previous example.

### Example 13

A non-decomposable matrix tablet for binging acetaldehyde in the stomach The relative composition comprises:

| | | |
|---|---|---|
| Composition | 25 | portions |
| Eudragit RS | 20 to 30 | portions |
| Microcrystalline cellulose | 20 to 50 | portions |

Tablets that contain 100 to 200 mg of cysteine are compressed from a powder mixture by means of the equipment conventionally used in the drug industry. The tablet is a monolithic matrix tablet that does not decompose in the stomach. The effective substance is released and dissolves in the gastric juice in a prolonged manner, resulting in a prolonged binding effect of the acetaldehyde. Being a binding substance insoluble in water, Eudragit RS can be replaced with pharmaceutical additives (such as ethylcellulose) that act in a corresponding manner.

### Example 14

A Film-Coated Tablet for Binding Acetaldehyde in the Stomach

Pharmaceutical formulations that release acetaldehyde-binding compounds into the stomach in a prolonged manner can also be manufactured as tablets, which are coated with a porous film. The core of the composition contains:

| | | |
|---|---|---|
| Cysteine (20 to 50 portions) | 30 | portions |
| Lactose | 50 to 80 | portions |
| Magnesium stearate | 1 to 2 | portions |
| Talcum | 1 to 2 | portions |

A powdery mixture is compressed into tablets, which are coated with a film, using the techniques generally used in the drug industry, for example:

| Ethylcellulose | 2 to 5 | portions |
|---|---|---|
| Hydroxypropyl methylcellulose (HPMC) | 1 to 2 | portions |
| Ethanol | 95 | portions |

Ethylcellulose does not dissolve in the gastro-intestinal duct, but HPMC does, forming pores in the film and allowing the cysteine to release from the tablet in a prolonged manner.

### Example 15

Non-decomposable granules for a prolonged release of the acetaldehyde-binding compounds into the stomach

The relative composition contains, for example:

| | | |
|---|---|---|
| Cysteine | 25 | portions |
| Eudragit RS or ethylcellulose | 20 to 30 | portions |
| Microcrystalline cellulose | 40 to 60 | portions |

| | |
|---|---|
| Ethanol | q.s. |

Powdery substances were mixed and moistened with ethanol by the equipment generally used in the drug industry. The moistened mixture is granulated and dried by any known methods. If necessary, the formed matrix granules can be coated with a hydroxypropyl methylcellulose film of a low molecular weight to cover the taste of cysteine. A sufficient number of granules containing one dose of cysteine (100 to 200 mg) can be portioned out into gelatine capsules or compressed into tablets with microcrystalline cellulose, for example.

1-21. (canceled)

22. An acetaldehyde-binding compound for use in reducing tobacco and/or alcohol dependence, wherein the compound is to be used simultaneously with consuming a tobacco product and/or with consuming alcohol, and wherein the acetaldehyde-binding compound is selected from a group comprising: L-cysteine, D-cysteine, cysteinic acid, cysteine-glycine, cystine, threo- or erythro-β-phenyl-DL-cysteine, β-tetramethylene-DL-cysteine, methionine, serine, D-penicillamine or its N-terminal dipeptide, semicarbazide, glutathione, reduced glutathione, β-mercaptoethyl amine, DL-homocysteine, DL-homocysteinic acid, N-acetylcysteine, L-cysteinyl-L-valine, β-β-tetramethylene-DL-cysteine, cysteinylglycine, mercaptoethyl glycine, cysteine hydrochloride, thiamine hydrochloride, sodium metabisulphite, arginine, glycine, lycine, ammonium chloride, 1,4 ditiothreitol, mercaptane, or it is the salt of any of these compounds.

23. A compound according to claim 22 for use in reducing tobacco and/or alcohol dependence, characterized in that
a) a preparation comprising the compound capable of binding acetaldehyde is given to an individual, who is dependent on tobacco or alcohol, to be used simultaneously with consuming a tobacco product and/or alcohol, and/or
b) a tobacco product is given to an individual, who is dependent on tobacco or alcohol, to which tobacco product a part or a component is attached, comprising the compound that is capable of binding acetaldehyde during or in connection with smoking and/or consuming alcohol, and
c) the individual is allowed to smoke and/or drink alcohol, whereby the acetaldehyde-binding compound binds the acetaldehyde formed from the tobacco product or alcohol, thus preventing the formation of a compound called harmane, and
d) the stages (a) or (b), or both simultaneously, are repeated, and (c) as many times that the reduction in the tobacco and/or alcohol dependence of the individual results in a cessation of smoking or consuming alcohol.

24. A compound according to claim 22, characterized in that the tobacco product refers to a cigarette, cigar or pipe.

25. A compound according to claim 22, characterized in that the use comprises a stage, wherein the individual is told about the carcinogenity and other health hazards caused by the acetaldehyde produced by tobacco or alcohol in the mouth or elsewhere in the digestive system.

26. A compound according to claim 22, characterized in that the preparation that is kept in the mouth releases the acetaldehyde-binding compound during the consumption of at least one tobacco product.

27. A compound according to claim 22, characterized in that the preparation that is kept in the mouth releases the acetaldehyde-binding compound for at least 5 minutes.

28. A compound according to claim 22, characterized in that the preparation that is kept in the mouth comprises 1 to 20 mg of the acetaldehyde-binding compound.

29. A compound according to claim 22, characterized in that the preparation that is kept in the mouth releases the acetaldehyde-binding compound for at least half an hour.

30. A compound according to claim 29, characterized in that the preparation that is kept in the mouth comprises at least 50 mg of the acetaldehyde-binding compound.

31. A compound according to claim 22, characterized in that the individual is given, to be consumed in connection with using alcohol or as long as there is alcohol in blood, a preparation that releases the acetaldehyde-binding compound into the stomach for at least 30 minutes.

32. A compound according to claim 31, characterized in that the individual is given a preparation at intervals of 2 to 4 hours.

33. A compound according to claim 31, characterized in that the individual has an acid-free stomach, a Helicobacter infection, or the individual uses drugs that reduce the secretion of gastric acid.

34. A compound according to claim 22, characterized in that the part or component comprises 0.5 to 100 mg of the acetaldehyde-binding compound.

35. A compound according to claim 22, characterized in that the acetaldehyde-binding compound comprises one or more free amino groups and sulphhydryl or sulphonic groups.

36. A compound according to claim 22, characterized in that the acetaldehyde-binding compound comprises one or more compounds according to the formula (I) 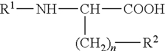
wherein
R¹ is hydrogen or an acyl group that has 1 to 4 carbon atoms;
R² is a sulphhydryl or sulphonic group; and
n is 1, 2, 3 or 4,
or a salt of these compounds.

37. A compound according to claim 22, characterized in that the acetaldehyde-binding compound is selected from a group comprising: L- or D-cysteine, acetyl cysteine, N-penicillamine, or the derivatives of cysteine that function in the same way as L- or D-cysteine, or the salts of these compounds.

38. A compound according to claim 22, intended for the reduction in tobacco and/or alcohol dependence, characterized in that the method comprises a stage(s), wherein acetaldehyde-binding compounds are used for the withdrawal of an individual from tobacco, or a stage(s), wherein nicotine-replacement products are used for the withdrawal of an individual from tobacco.

39. A compound according to claim 22, intended for the reduction in tobacco and/or alcohol dependence, characterized in that the preparations comprising the acetaldehyde-binding compound, and the components, which are attached to the tobacco product and which comprises the compound that is capable of binding acetaldehyde during smoking, are used during the period of time when the individual is not able to refrain from smoking, and the nicotine replacement products are used during the period of time when the individual is able to refrain from smoking.

40. The use of acetaldehyde-binding compounds for the manufacture of the preparation or the part or component, which is attached to the tobacco product and which is capable of binding acetaldehyde, for a reduction in tobacco and/or alcohol dependence.

41. A method of reducing tobacco and/or alcohol dependence, according to which
a) an individual, who is dependent on tobacco or alcohol, is given a preparation comprising the compound capable of binding acetaldehyde to be consumed simultaneously with consuming a tobacco product and/or alcohol, and/or
b) the individual, who is dependent on tobacco or alcohol, is given a tobacco product, to which a part or component is attached, comprising the compound capable of binding acetaldehyde during smoking and/or drinking alcohol, and
c) the individual is allowed to smoke and/or drink alcohol, whereby the acetaldehyde-binding compound binds the acetaldehyde formed from the tobacco product or alcohol, thus preventing the formation of a substance called harmane, and
d) the stages (a) and (b), or both simultaneously, are repeated, and (c) as many times that the reduction in the smoking and/or alcohol dependence of the individual results in a cessation of smoking or consuming alcohol.
